(19) United States
(12) Patent Application Publication
(10) Pub. No.: US 2022/0022911 A1
(43) Pub. Date: Jan. 27, 2022

(54) Scalpel with lighting

(71) Applicant: Jaewoong Lee, Fairfax, VA (US)

(72) Inventor: Jaewoong Lee, Fairfax, VA (US)

(21) Appl. No.: 17/300,692

(22) Filed: Oct. 1, 2021

(51) Int. Cl. A61B 17/3211; A61B 90/30

(52) U.S. Cl. CPC A61B 17/3211; A61B 90/30

(57) ABSTRACT

The present invention relates to a scalpel with lighting for a surgical instrument, and more specifically, to a scalpel with lighting that facilitates surgery by illuminating the necessary places with a partial light source where the light from the lighting lamp is not directly irradiated into the body, that is, a curved place inside a human body when performing delicate procedures such as suturing and cutting of blood vessels or nerves in areas where the light source is difficult to reach during organ extraction. The scalpel with lighting comprising: a cylindrical housing in which a pluralities of batteries inserted, and an on-off switch formed at the rear side thereof; a cap portion screwed to the front of the cylindrical housing, in which an LED bulb is inserted, and the front end being opened; and a fixture for fixing the blade inside the cap portion; wherein the light from the LED bulb is configured to illuminate the operation site through the open front end the cap portion.

### TECHNICAL FIELD

The present invention relates to a scalpel with lighting for a surgical instrument, and more specifically, to a scalpel with lighting that facilitates surgery by illuminating the necessary places with a partial light source where the light from the lighting lamp is not directly irradiated into the body, that is, a curved place inside a human body when performing delicate procedures such as suturing and cutting of blood vessels or nerves in areas where the light source is difficult to reach during organ extraction.

### BACKGROUND

When a medical operation is performed in a hospital, lighting is essential for accuracy and safety, and lighting devices such as shadowless surgical lamps are generally provided as a light sources for lighting used in an operating room. In addition, a lighting device in the form of a head lamp worn by a doctor is also used to illuminate a local area.

Even though these lighting devices are easy to use when the operation site is opened and illuminated from outside, but the light source cannot directly irradiate during incision surgery deep inside the body or during local incisional surgery or lateral surgery, resulting in shadows, and accordingly, the area to be treated becomes relatively dark.

In particular, during organ transplantation and extraction, the light does not reach the inside of the body, so there is a hassle in that an assistant must move a separate lighting device according to the operator's verbal instructions. In addition, since the operator could lose concentration during an operation for a long time, intensive illumination is required where the blade or the surgical instrument could touch when performing the procedure in a very narrow area inside the body using a surgical blade or an electric surgical device. To prepare for this, the head of the operator must be moved to use the light source of the head lamp, and the use of lighting with the assistance of an assistant may affect long-term surgery, such as poor accuracy of surgical operations or delays in delicate surgical operations that compete for vision.

In order to solve this problem, Fiberoptic Light has been developed and used as an instrument capable of irradiating light even during surgery in a narrow and deep area. Fiberoptic Light is a lighting device that uses fiberoptic to supply light to a narrow and deep affected area.

In this method, since a halogen lamp is used as a separate light source in the light source supply device, considerable heat is generated. In addition, the light source supply device and the surgical instrument are connected by an optical cable, and since the cable is thick and a long optical cable is used due to the sheath and connector to protect the optical cable, it is inconvenient to connect each time in use and during operation. In addition to the operator, an assistant must always be present, and there is also the inconvenience that the assistant must always be careful not to let the optical cable touch the patient's affected part or other surgical instruments during use.

And, to prevent contamination or transmission, surgical instruments must be sterilized with gamma rays or the like before use due to the high level of cleanliness required.

### SUMMARY

The present invention has been proposed to solve the above problems, and by using it directly coupled to a surgical tool such as an electric surgical instrument or a surgical blade holder, it can be safely and efficiently operated by irradiating light even during surgery in a narrow and deep area.

In an embodiment, a scalpel with lighting of the present invention comprises:

a cylindrical housing in which a pluralities of batteries inserted, and an on-off switch formed at the rear side thereof;

a cap portion screwed to the front of the cylindrical housing, in which an LED bulb is inserted, and the front end being opened; and

a fixture for fixing the blade inside the cap portion;

wherein the light from the LED bulb is configured to illuminate the operation site through the open front end the cap portion.

### BRIEF DESCRIPTION OF THE DRAWINGS

The above and other aspects, features, and advantages of certain preferred embodiments of the present disclosure will be more apparent from the following description taken in conjunction with the accompanying drawings, in which:

FIG. 1A is a view showing a scalpel with light according to an embodiment of the present disclosure.

FIG. 1B is an exploded view showing a scalpel with light according to an embodiment of the present disclosure in FIG. 1A.

FIG. 2 is a rear view showing a configuration of a fixture for a blade by which a blade is fixed inside a cap portion according to an embodiment of the present disclosure.

FIG. 3 is a view illustrating a fixture for a blade according to an embodiment of the present disclosure in FIG. 2.

### DETAILED DESCRIPTION

Technical problems achieved by the present invention and implementation of the present invention will become clearer by the preferred embodiment described below. The following example is simply exemplified to explain the present invention and is not intended to limit the scope of the present invention. In addition, in the embodiment of the present invention, the same reference numerals are used for technical components that perform the same functions.

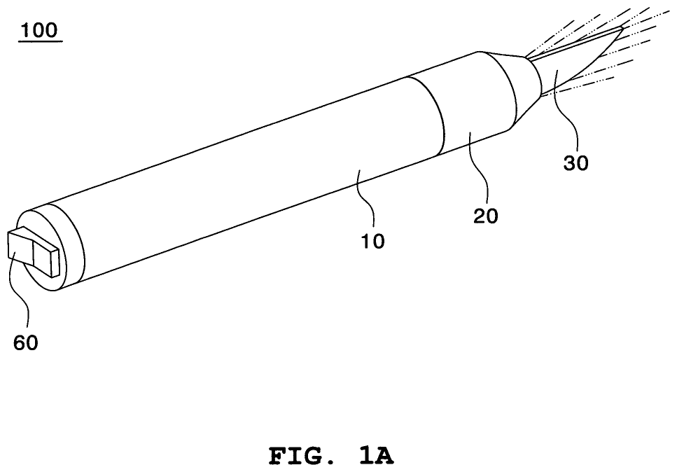

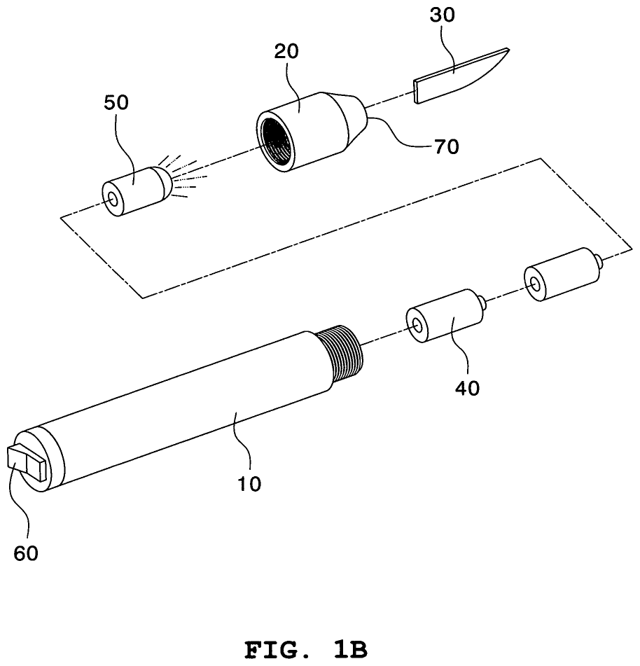

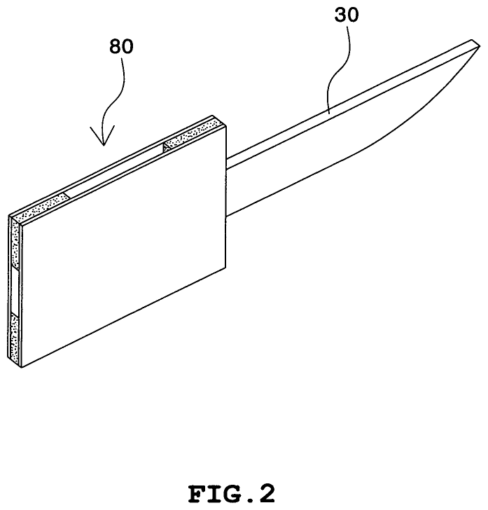

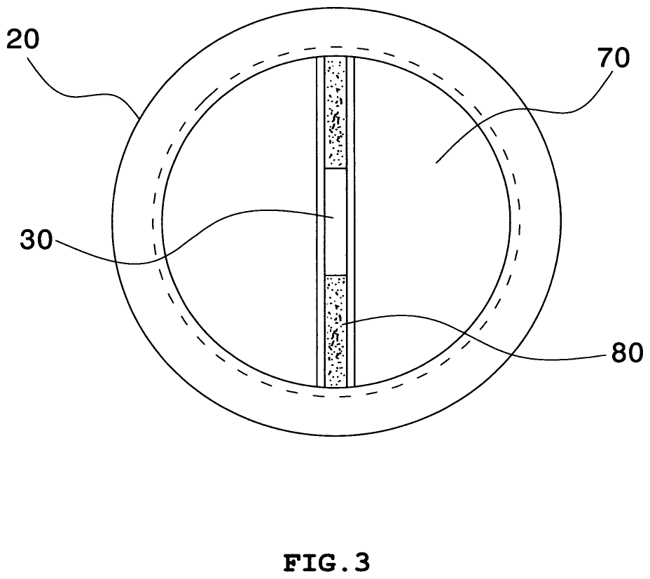

FIG. 1A is a view showing a scalpel with light according to an embodiment of the present disclosure. FIG. 1B is an exploded view showing a scalpel with light according to an embodiment of the present disclosure in FIG. 1A. FIG. 2 is a rear view showing a configuration of a fixture for a blade by which a blade is fixed inside a cap portion according to an embodiment of the present disclosure. FIG. 3 is a view illustrating a fixture for a blade according to an embodiment of the present disclosure in FIG. 2.

Referring to FIGS. 1 and 2, the scalpel with lighting 100 of the present invention is composed of a cylindrical housing 10 and a cap portion 20. More specifically, the cylindrical housing 10 and cap portion are connected by screwing each other and made of the same material surrounding the batteries 40, and a LED lamp 50.

The batteries are for supplying power to the LED lamp 50 and are usual AAA type battery in the embodiment of the present invention. The battery generally outputs a voltage of 1.5V, and in the present invention, two or more batteries are connected in series to drive the LED lamp 30.

The above-described housing 10 and the cap portion 20 are made of a polymer material or a flexible plastic material. Each electrode terminals of the LED lamp 50 are electrically connected to the anodes and the cathodes of the batteries 40.

Inside the cap portion, the LED lamp 50 is inserted from the rear part, and there is a fixture for fixing a blade inside the cap portion. The front end 70 of the cap portion 20 is opened, therefore, when the LED lamp 50 is turned-on, the lay of light delivered from the front end 70 of the cap portion 20 and can illuminate the operation site.

The cylindrical housing has an on-off switch formed at the rear side thereof, therefore, a user can on and off the LED lamp 50.

Inside the cap portion 20, there is a fixture for fixing the blade inside the cap portion 20. Referring FIGS. 2 and 3, the height of the fixture 80 substantially corresponds to the diameter of the cap portion 20, and the fixture 80 is made of two panel-like members and four small square-like members. The four square-like members are sandwiched between the two panel-like members and the width of the square-like members corresponds to that of the blade 30. And the distance between two square-like members in vertical direction corresponds to that of the height of the blade 30. Therefore, a blade 30 can be fixed between two panel like members and does not move because of four square-like members.

When an operation starts, an operator turn on the switch 60 to light the LED lamp 50, then a ray of light go through the front end of the cap portion 20 and an operator can light the operation site and does the operation.

As described above, preferred embodiments of the present invention have been described, but the rights of the present invention are not limited to the above-described embodiments, and are defined by the claims, and common knowledge in the technical field of the present invention.

What is claimed is:

1. A scalpel with lighting comprising:
a cylindrical housing in which a pluralities of batteries inserted, and an on-off switch formed at the rear side thereof;
a cap portion screwed to the front of the cylindrical housing, in which an LED bulb is inserted, and the front end being opened; and
a fixture for fixing the blade inside the cap portion;
wherein the light from the LED bulb is configured to illuminate the operation site through the open front end the cap portion.
